(19) United States
(12) Patent Application Publication
(10) Pub. No.: US 2012/0158050 A1
(43) Pub. Date: Jun. 21, 2012

(54) SUTURE REPAIRING SYSTEM AND METHOD

(75) Inventor: Michael Warren Woods, Missoula, MT (US)

(73) Assignee: MEDevice IP Holdings, LLC.

(21) Appl. No.: 13/297,927

(22) Filed: Nov. 16, 2011

Related U.S. Application Data
(60) Provisional application No. 61/423,197, filed on Dec. 15, 2010.

(51) Int. Cl. A61B 17/04

(52) U.S. Cl. 606/232

(57) ABSTRACT

A suture repair system and method for repairing a planned or unplanned opening in a human tissue plane. A handle having a deployable working end accommodates a re-loadable cartridge containing a suture and anchor in order to form an anchored limb at an edge of the tissue to be repaired. An end plate at a distal end of the suture cartridge slips into the tissue edge and deploys an anchor under the edge of the tissue that receives and prevents the suture from backing out. The handle can be shifted to an opposite edge of the tissue and sutured. A closure clip further joins the two anchored limbs of the suture via a one-way locking mechanism. An approximating tool pushes down the clip towards the defected tissue in order to achieve appropriate tissue approximation.

### CROSS-REFERENCE TO PROVISIONAL PATENT APPLICATION

This nonprovisional patent application claims the benefit under 35 U.S.C. §119(e) of U.S. Provisional Application Ser. No. 61/423,197 entitled, “Suture Repairing System and Method,” which was filed on Dec. 15, 2010 and is incorporated herein by reference in its entirety.

### TECHNICAL FIELD

Embodiments are generally related to surgical instruments and techniques. Embodiments are additionally related to suture repair systems and methods.

### BACKGROUND OF THE INVENTION

Repair of planned or unplanned openings in deeply located tissue planes is frequently required during minimally invasive or arthroscopic surgical procedures. A suture repair device can be employed for re-approximating an inadvertently opened tissue plane or closing a planned surgical incision in a tissue plane. Such a device is designed to be employed in a tight space such as, for example, an endoscopic tubular retractor, where conventional closure methods are problematic or impossible utilizing traditional suturing techniques.

Conventional suture repair systems permit a surgeon to perform a typical side-to-side rotational sewing maneuver that drives a needle and attaches suture through the tissue on each side of the opened area. The loose ends of the suture can be tied together to form a closed loop with a knot across the opening, thus re-approximating and closing the opening in the tissue plane. The tension and size of the resultant closed loop of suture is varied according to need, achieving the desired goal of closure of the tissue plane. In a tightly constrained environment, with limited access to and visualization of the target tissue, the ability to affect the above-described traditional maneuvers becomes very difficult or physically unachievable. Prior art techniques exist for some areas of the body where at least some freedom of movement is still possible. We have found no existing technology that can be used safely and effectively within the tightly constrained environment of a tubular retractor.

Based on the forgoing, it is believed that a need exists for an effective, safe tissue approximation system that provides a mechanism and method for obtaining secure closure of tissue planes encountered in the severely constrained environment of current minimally invasive tubular retractor systems, as will be described in greater detail herein.

### BRIEF SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

It is, therefore, one aspect of the disclosed embodiments to provide for improved surgical instruments and techniques.

It is another aspect of the disclosed embodiments to provide for an improved suture delivery system and method.

It is a further aspect of the disclosed embodiments to provide for an improved method of tissue approximation via secure closure of the suture across the tissue opening in a physically constrained environment.

The aforementioned aspects and other objectives and advantages can now be achieved as described herein. A suture delivery system and method for attaching the desired suture to affected tissues is disclosed herein. The disclosed device of such a system can be configured to include a handle with an integrated actuating mechanism attached. Such an approach can accommodate reloadable cartridges containing the suture and tissue anchor of desired sizes. The distal working end can be set against one edge of the tissue opening. The actuator can be deployed, placing the tissue anchor and attached suture into and through the tissue in a safe and secure fashion. Another suture cartridge can be loaded and the process repeated in the opposite side of the tissue opening. Such a procedure can be repeated along the tissue opening sequentially until the desired number of sutures/anchors is achieved. A closable dip can then be attached to the two free ends of the anchored suture opposite each other. A separate approximating tool can then be employed to secure the two pieces of suture together securely, thus dosing the defect in the tissue with the desired amount of tension. The second device can then trim the excess suture.

The disclosed suture repair system and method permits a surgeon to approximate and repair tissue defects via an up-down motion approach in a constrained environment. The suture can be re-absorbable or non-absorbable material in varying sizes as the particular situation dictates. The closure clip can also be employed “stand alone;” used with existing bone anchor systems to facilitate a “knotless closure” construct. Such an approach can effectively repair tissues with rapid and secure approximation with a limited amount of operating clearance.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates a perspective view of a repair system, in accordance with the disclosed embodiments;

FIGS. 2-5 illustrate logical operational steps for repairing tissue openings utilizing the suture repair system depicted in FIG. 1, in accordance with the disclosed embodiments; and

FIG. 6 illustrates a high level flow chart of operations illustrating logical operational steps of a method for the repair of tissue openings, in accordance with the disclosed embodiments.

### DETAILED DESCRIPTION

The particular values and configurations discussed in these non-limiting examples can be varied and are cited merely to illustrate at least one embodiment and are not intended to limit the scope thereof.

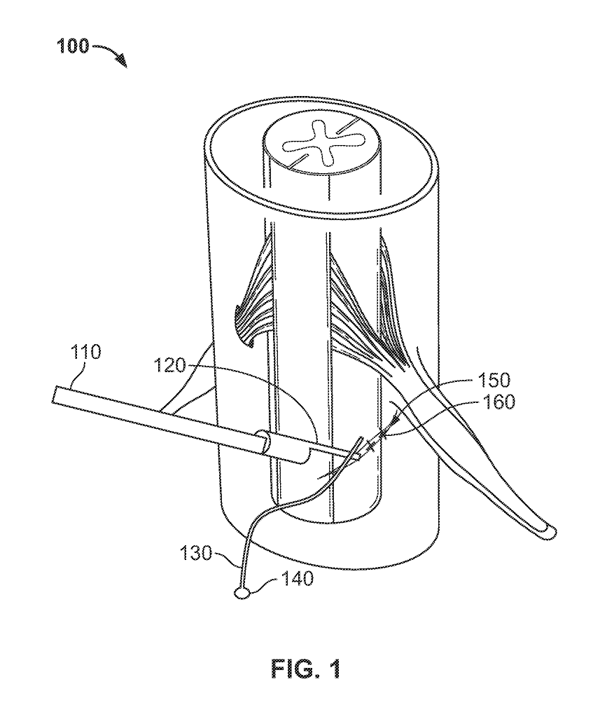

FIG. 1 illustrates a perspective view of a suture repair system 100, in accordance with the disclosed embodiments. The suture repair system 100 can be effectively employed in a wide range of surgical applications such as, for example, but not limited to, minimal invasive surgical procedures and arthroscopic surgeries for closing incisions or other planned or unplanned openings in body tissue planes 150. The system 100 can be generally employed to place multiple sutures 160 in the edges 210 and 220 of the tissue opening 150. The suture repair system 100 typically enables a surgeon to approximate and repair the tissue via an up-down motion approach. Note that the technique used for repairing the tissue 150 will depend upon the size, nature, and ability to access and visualize the area of interest.

The suture repair system 100 generally includes an handle/actuator 110, a cartridge 120 containing the suture 130, an approximating tool 370, a closure clip 360, and an integrated cutter (not shown). The handle 110 will contain an ergonomically designed actuator designed to retain the reloadable cartridges 120 containing the suture and anchor 130. The distal end of the cartridge can be inserted into the tissue opening and placed against the edge of the tissue. The actuator can be deployed and the suture is passed through the tissue and secured to the anchor on the underside of the tissue edge. The now secured suture is then stripped out of the cartridge and retained until the closure 135. The actuator 110 is then reloaded with another suture cartridge and the process repeated on the opposite side of the tissue opening 220.

The closure clip 360 is threaded onto the two free suture ends and then into the approximating tool 370. The approximating tool 370 pushes down the clip 360 towards the tissue to be repaired 150, while appropriate back tension is applied to the free ends of the suture. This then brings the separated ends of the tissue together, thus re-approximating the opening 150. The integrated cutter trims the excess portions of the suture on reaching a proper closure tension. The suture 135 can be of appropriate size and material as the particular situation dictates. Several types of re-absorbable and permanent sutures can be available. Monofilament or braided types of suture will also be available. Each type of suture can be contained in separate cartridges with appropriate sized anchors for each.

The suture repair system 100 described herein is intended as an example and not as an architectural limitation with respect to particular embodiments. Such embodiments, however, are not limited to any particular surgical procedure and technique. The surgical tools and instruments described herein above may be provided in various combinations as kits for facilitating not only the distribution of the surgical tools and instruments but also the deployment and utilization of the surgical tools and instruments in a wide range of endoscopic surgical procedures including arthroscopy, laparoscopy, gastroendoscopy, and other varying types of limited access surgical platforms.

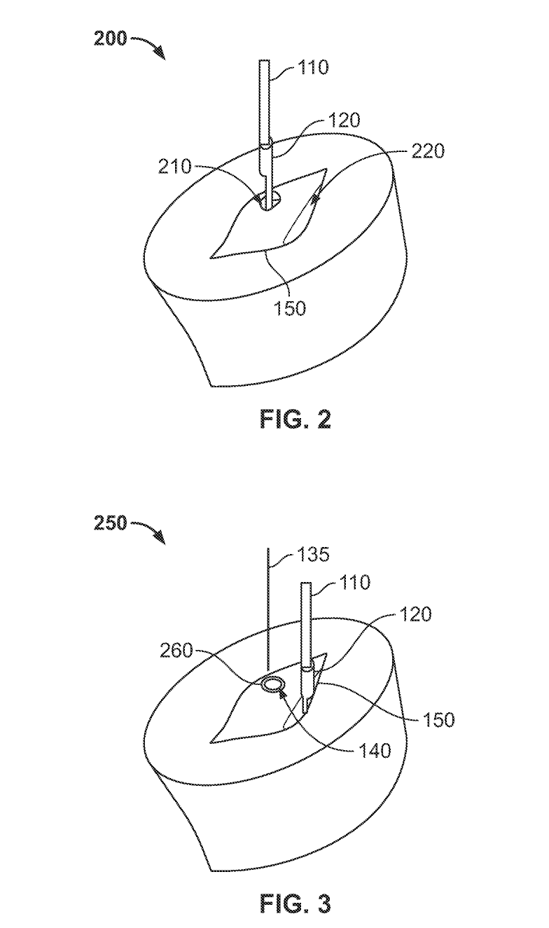

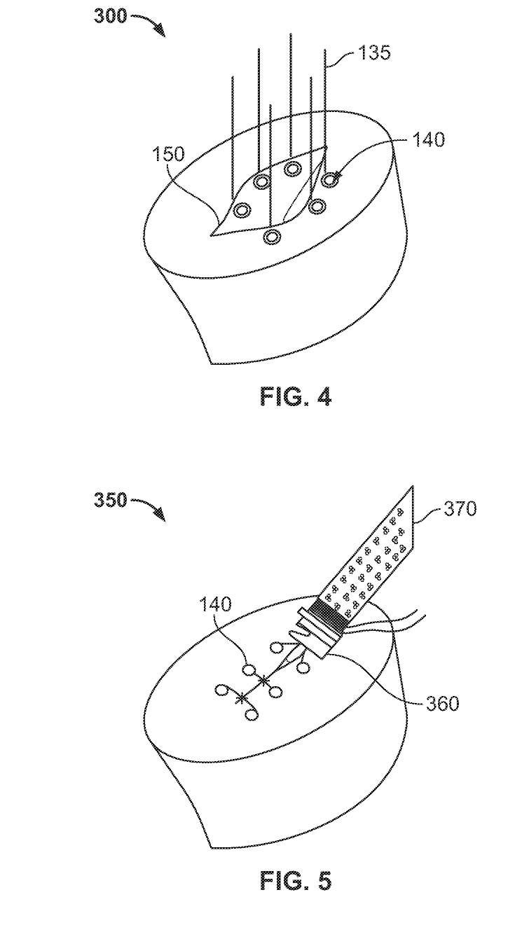

FIGS. 2-5 illustrate logical operational steps 200, 250, 300, and 350 for suture repair of tissue plane openings 150 utilizing the suture repair system 100 discussed earlier, ire accordance with the disclosed embodiments. Note that in FIGS. 1-6, identical or similar parts are generally indicated by identical reference numerals. The suture repair system 100 can be employed to obtain a rapid and secure tissue approximation and ligation in varying surgical procedures. In general, approximation of tissues typically involves the placement of one or more sutures 160 in order to pull the edges 210 and 220 of the tissue 150 together to affect the desired closure.

Note that the closure clip 360 can also be utilized in conjunction with a bone anchor system to make a simple “quick tie” closure. The closure clip 360 can be pushed towards the tissue in order to achieve the closure tension. The excess portion of the suture 130 can thereafter be trimmed utilizing the integrated cutter, as depicted in FIG. 5.

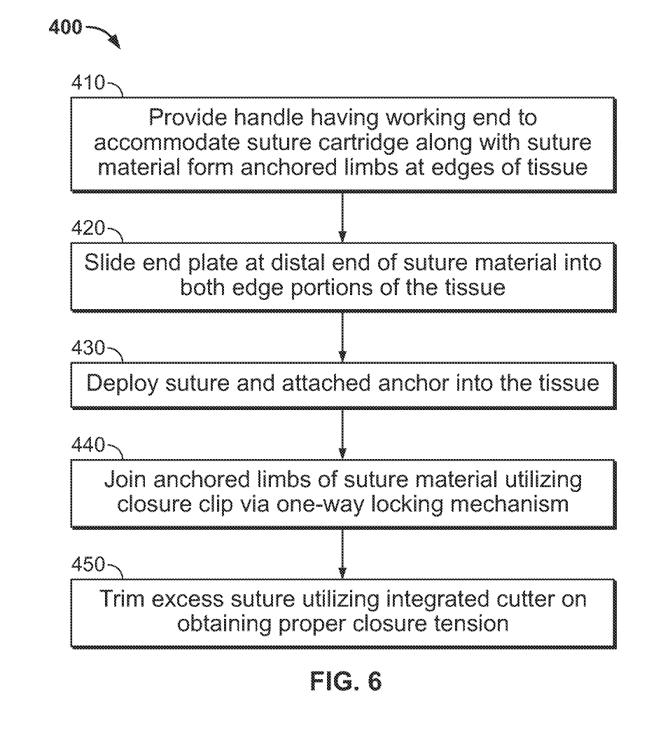

FIG. 6 illustrates a high level flow chart of operations illustrating logical operational steps of method 400 for repairing, for example, the tissue opening 150 in accordance with the disclosed embodiments. Again as a reminder, note that in FIGS. 1-6, identical or similar parts are generally indicated by identical reference numerals. As indicated at block 410, the handle 110 having the deployable working end can be provided to accommodate the cartridge 120 containing the suture and anchor 130 in order to form the anchored limbs 135 at both edge portions 210 and 220 of the tissue 150. Thereafter, as indicated at block 420, the end plate 140 at the distal end of the suture cartridge 130 can be slipped into the edge portions 210 and 220 of the tissue 150.

Next, as described at block 430, the suture cartridge 120 along with the end plate 140 can be further received and prevented from backing out of the tissue 150 via the anchor 260, That is, the operation depicted at block 430 describes a procedure in which the suture can be deployed and the anchor described herein attached into the tissue. Thereafter, as illustrated in block 440, the anchored limbs 135 at the edges 210 and 220 of the tissue 150 can be joined utilizing the closure clip 360 via the one-way locking mechanism described herein.

Note that the closure clip 360 can be pushed towards the tissue 150 via the approximating tool 370. The closure clip 360 can bring the opposite edges 210 and 220 of the tissue closer by knotting the anchored limbs 135 in order to obtain a secure closure. The approximating tool 370 further pushes the closure clip 360 towards the tissue 150 in order to ensure better positioning of the closure clip 360 with respect to the tissue 150. Finally, the excess portion of the suture 130 can be trimmed via the integrated cutter, as depicted at block 450. Such a system 100 and/or a method 500 can be therefore effectively employed to repair tissues in a wide range of surgical applications, particularly in the context of a limited access surgical environment.

Based on the foregoing, it can be appreciated that the disclosed systems and methods can be implemented in the context of varying embodiments. For example, in one embodiment, a suture repair system can be configured to include, but not limited to, an ergonomic handle having a deployable working end that accommodates a cartridge containing a suture and anchor in order to form a pair of anchored limbs at both edges of an opening in a tissue plane. The deployed anchor and the suture together comprise an anchored suture.

Such a system can also include, in some embodiments, an end plate at a distal end of the suture cartridge that is placed against the edge of the tissue, a deployable anchor positioned under the edge and for receiving and preventing the suture from backing out of the tissue, a closure clip that joins the pair of anchored limbs of the sutured material via a one-way locking mechanism, and therefore effectively repair an opening of the tissue in a limited access surgical environment. In some embodiments of such a system, an integrated cutter that can also be employed for trimming the excess portion of the aforementioned anchored suture.

All or or one or more of the aforementioned features of such a system can thus be employed to effectively repair the opening of the aformenentioned tissue in the context of, for example, a limited access surgical environment. In other embodiments of such a system, an approximating tool can be employed, which pushes down the clip and towards the tissue in the tissue plane in order to achieve appropriate tissue approximation and closure thereof while maintaining an appropriate back tension on the anchored sutures.

In still other embodiments of such a system, the aforementioned handle can be an actuator. Additionally, the end plate can be positioned at a distal end of the suture cartridge. As indicated previously, such suture cartridge can be configured from, for example, a slow re-absorbable type material or a non-absorbable material. Additionally, the aforementioned cartridge can be configured as a re-loadable cartridge.

In other embodiments, a method can be implemented for suture repair of a planned or unplanned tissue opening. Such a method can include, for example, providing a handle having a deployable working end to accommodate a cartridge containing a suture in order to form a pair of anchored limbs at both edge portions of a tissue; sliding an end plate at a distal end of the suture cartridge into the edge portions of the tissue in order to receive and prevent the suture from backing out of the tissue via a deployable tissue anchor; and joining the pair of anchored limbs of the sutured material utilizing a closure clip via a one-way locking mechanism in order to thereafter trim an excess portion of the suture via an integrated cutter to effectively repair an opening of the tissue in a limited access surgical environment.

In other embodiments of such a method, an operation can be implemented for trimming the excess portion of the suture by pushing down the clip towards the opening of the tissue opening an approximating tool. Additionally, in some embodiments, an operation can be implemented for positioning the end plate at a distal end of the suture cartridge. In yet other embodiments (or the same embodiments) of such a method, an operation can be implemented for approximating and repairing the tissue via an up-down motion approach. In still other embodiments (or the same embodiment), an operation can be implemented for placing the closure clip in conjunction with a bone anchor unit to form a simple quick tie closure. Additional embodiments (or the same embodiment) can include, for example, an operation for placing the suture cartridge at regular intervals in order to ensure complete closure across the tissue in a complex surgical procedure.

It will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

What is claimed is:

1. A suture repair system, comprising:
an ergonomic handle having a deployable working end that accommodates a cartridge containing a suture and anchor in order to form a pair of anchored limbs at both edges of an opening in a tissue plane, said anchored limbs and said suture comprising an anchored suture;
an end plate at a distal end of said suture cartridge that slips into said edges of said tissue;
a deployable anchor positioned under said edges and for receiving and preventing said suture from backing out of said tissue; and
a closure clip that joins said pair of anchored limbs of said sutured material via a one-way locking mechanism and therefore effectively repair an opening of said tissue in a limited access surgical environment.

2. The system of claim 1 further comprising:
an integrated cutter for trimming an excess portion of said anchored suture.

3. The system of claim 1 further comprising an approximating tool that pushes down said clip and towards said tissue in said tissue plane in order to achieve appropriate tissue approximation and closure thereof while maintaining an appropriate back tension on said anchored sutures.

4. The system of claim 1 wherein said handle comprises an actuator.

5. The system of claim 1 wherein said end plate is positioned at a distal end of said suture cartridge.

6. The system of claim 1 wherein said suture cartridge comprises a slow re-absorbable type material.

7. The system of claim 1 wherein said suture cartridge comprises a non-absorbable material.

8. The system of claim 1 wherein said cartridge comprises a re-loadable cartridge.

9. A suture repair system, comprising:
an ergonomic handle having a deployable working end that accommodates a cartridge containing a suture and anchor in order to form a pair of anchored limbs at both edges of an opening in a tissue plane, said anchored limbs and said suture and anchor comprising an anchored suture;
an end plate at a distal end of said suture cartridge that slips into said edges of said tissue;
a deployable anchor positioned under said edges and for receiving and preventing said suture from backing out of said tissue;
a closure dip that joins said pair of anchored limbs of said sutured material via a one-way locking mechanism; and
an integrated cutter for trimming an excess portion of said anchored suture, wherein said ergonomic handle, said end plate, said deployable anchor, said close clip, and said integrated cutter effectively repair an opening of said tissue in a limited access surgical environment.

10. The system of claim 9 further comprising an approximating tool that pushes down said clip and towards said tissue in said tissue plane in order to achieve appropriate tissue approximation and closure thereof while maintaining an appropriate back tension on said anchored sutures.

11. The system of claim 9 wherein said handle comprises an actuator.

12. The system of claim 9 wherein said end plate is positioned at a distal end of said suture cartridge.

13. The system of claim 9 wherein said suture cartridge comprises a slow re-absorbable type material or a non-absorbable material.

14. The system of claim 9 wherein said cartridge comprises a re-loadable cartridge.

15. A method for suture repair of a planned or unplanned tissue opening, said method comprising:
providing a handle having a deployable working end to accommodate a cartridge containing a suture and anchor in order to form a pair of anchored limbs at both edge portions of a tissue;
sliding an end plate at a distal end of said suture cartridge into said edge portions of said tissue in order to receive and prevent said suture from backing out of said tissue via a deployable tissue anchor; and
joining said pair of anchored limbs of said sutured material utilizing a closure clip via a one-way locking mechanism in order to thereafter trim an excess portion of said suture via an integrated cutter to effectively repair an opening of said tissue in a limited access surgical environment.

16. The method of claim 15 further comprising trimming said excess portion of said suture by pushing down said clip towards said opening of said tissue opening an approximating tool.

17. The system of claim 15 further comprising positioning said end plate at a distal end of said suture cartridge.

18. The method of claim 15 further comprising approximating and repairing said tissue via an up-down motion approach.

19. The method of claim 15 further comprising placing said closure clip in conjunction with a bone anchor unit to form a simple quick tie closure.

20. The method of claim 15 further comprising placing said suture cartridge at regular intervals in order to ensure complete closure across said tissue in a complex surgical procedure.
